(19) United States
(12) Patent Application Publication
(10) Pub. No.: US 2021/0023320 A1
(43) Pub. Date: Jan. 28, 2021

(54) LARYNGEAL MASK AIRWAY

(71) Applicant: AJOU UNIVERSITY INDUSTRY-ACADEMIC COOPERATION FOUNDATION, Suwon-si, Gyeonggi-do (KR)

(72) Inventors: In Kyong YI, Suwon-si, Gyeonggi-do (KR); Yun Jeong CHAE, Suwon-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 16/979,862

(22) PCT Filed: Mar. 14, 2019
(86) PCT No.: PCT/KR2019/002931

§ 371 Date: Sep. 11, 2020

(30) Foreign Application Priority Data
Mar. 14, 2018 (KR) 10-2018-0029826

(51) Int. Cl. A61M 16/04; A61B 1/267

(52) U.S. Cl. CPC A61M 16/049; A61B 1/2676

(57) ABSTRACT

A laryngeal mask airway includes: a guide tube that has one proximal end portion inserted into the larynx inside the trachea and the other distal end portion exposed to the outside of the trachea, guides the insertion of a flexible bronchoscope and an endotracheal tube inserted into the trachea, and is divided into left and right sides; a mask that is integrally connected to the one proximal end portion of the guide tube, prevents fluid and air from flowing into the larynx inside the trachea from the outside, and is divided into the left and right sides; a handle portion that is provided on the other side of the guide tube and protrudes to both sides of the guide tube to correspond to each other; and a coupling member that couples the guide tube and mask, which are divided into the left and right sides, to each other.

### TECHNICAL FIELD

The present invention relates to a laryngeal mask airway, and more specifically, to a laryngeal mask airway which guides the insertion of an endotracheal tube into the trachea, and is divided into left and right sides to be detached after being inserted into the trachea.

### BACKGROUND

In general, as a method capable of being used in a difficult airway situation where endotracheal intubation with a laryngoscope is not possible, there is an endotracheal intubation method using a flexible bronchoscope. It has been reported that this method significantly increases a success rate of the endotracheal intubation in any difficult airway situation. However, the endotracheal intubation method using the flexible bronchoscope is a difficult technique and considerable experience is required to learn the technique, so that it has a limitation that there are few clinicians who can implement it even though it is a good method.

The biggest reason why the intubation in the trachea by using the flexible bronchoscope is difficult is that the bronchoscope in the oral cavity is wandering without finding its direction. To compensate for this, there are a variety of airways that can provide a path through which the flexible bronchoscope passes from the lips to the epiglottis, and among them, it is known that the laryngeal mask airway most certainly provides the path. When such a laryngeal mask airway is inserted into the oral cavity and the flexible bronchoscope is inserted through the lumen, even a clinician with little experience can easily find the entrance of the airway.

In a case in which the flexible bronchoscope is placed and passes through the laryngeal mask airway, and an endotracheal tube is inserted into the trachea, there is inevitably a restriction on a size of an inner diameter of the laryngeal mask airway. Since the tube must pass through the inner diameter of the laryngeal mask airway, an outer diameter of the tube must be smaller than the inner diameter of the laryngeal mask airway. In general, the most appropriate sized tube for adults does not pass through the laryngeal mask airway, and thereby the endotracheal tube having a size smaller than a required size must be inserted.

Another problem is that it is not possible to remove the laryngeal mask airway in a state in which the flexible bronchoscope is inserted. Therefore, alternatively, the laryngeal mask airway is removed after the endotracheal tube is inserted through the laryngeal mask airway. However, when the laryngeal mask airway is removed, the endotracheal tube inserted into the lumen of the laryngeal mask airway is short, so that there is a problem that the endotracheal tube is separated from the airway while the laryngeal mask airway is removed.

In this way, as the endotracheal tube within the trachea is separated, it is difficult or dangerous to remove the laryngeal mask airway, so that there is a case that the laryngeal mask airway is placed without being removed. However, there are many situations in which the laryngeal mask airway must be removed. For example, in a case in which the patient's posture needs to be changed or in critically ill patients who need long-term mechanical breathing, a pressure of the laryngeal mask airway continually applied to the patient's mouth, resulting in complications such as swelling. Therefore, the laryngeal mask airway needs to be removed.

The technique of a background of the present invention is Korean Patent Unexamined Publication No. 2008-0031211 (published on Apr. 8, 2008).

### SUMMARY OF INVENTION

### Technical Problem

The present invention is created to meet the above needs, and an object of the present invention is to provide a laryngeal mask airway which can be easily detached while preventing an endotracheal tube inserted into the trachea through a guide tube from being detached from the trachea.

### Solution to Problem

In order to achieve the above object, the present invention provides a laryngeal mask airway to be inserted into a patient in order to ensure that the patient's airway is open, including: a guide tube that has one proximal end portion inserted into the larynx inside the trachea and the other distal end portion exposed to the outside of the trachea, guides the insertion of a flexible bronchoscope and an endotracheal tube inserted into the trachea in order to ensure that an airway is open, and is divided into left and right sides; a mask that is integrally connected to the one proximal end portion of the guide tube, prevents fluid and air from flowing into the larynx inside the trachea from the outside, and is divided into the left and right sides; a handle portion that is provided on the other side of the guide tube and protrudes to both sides of the guide tube to correspond to each other such that an operator easily holds the guide tube; and a coupling member that couples the guide tube and mask, which are divided into the left and right sides, to each other.

In the laryngeal mask airway according to the present invention, a first cutting surface may be formed at a center of the guide tube in a longitudinal direction, the guide tube may include a left guide tube disposed on a left side with respect to the first cutting surface, and a right guide tube disposed on a right side with respect to the first cutting surface, the left guide tube and the right guide tube may be coupled and detached by the coupling member, and the left guide tube and the right guide tube may have corresponding shapes.

A second cutting surface may be formed at a center of the mask in a longitudinal direction, the mask may include a left mask disposed on a left side with respect to the second cutting surface, and a right mask disposed on a right side with respect to the second cutting surface, the left mask and the right mask may be coupled and detached by the coupling member, and the left mask and the right mask may have corresponding shapes.

The left guide tube and the left mask may be integrally formed, and the right guide tube and the right mask may be integrally formed, and the coupling member may include a coupling protrusion formed on a contact surface of the right guide tube and the right mask, and a coupling groove formed on a contact surface of the left guide tube and the left mask, and on which the coupling protrusion is mounted.

The coupling protrusion may have a triangular shape and protrude outward from the contact surface of the right guide tube and the right mask, and the coupling groove may have a shape corresponding to that of the coupling protrusion, and may be inserted into the contact surface of the left guide tube and the left mask.

The coupling protrusion may have a shape protruding outward from the contact surface of the right guide tube and the right mask, and the coupling groove may have a shape corresponding to that of the coupling protrusion, and may be inserted into the contact surface of the left guide tube and the left mask.

The coupling protrusion may protrude outward from the contact surface of the left guide tube and the left mask, and may have a shape in which a circular protrusion is formed at an end, and the coupling groove may have a shape corresponding to that of the coupling protrusion, and may be inserted into the contact surface of the right guide tube and the right mask.

### Advantageous Effects

In the laryngeal mask airway according to the present invention, the operator grasps the handle portion, and then the guide tube and the mask are divided into the left and right sides with respect to the first cutting surface and the second cutting surface, so that in a case in which the tube is inserted while maintaining the placement of the flexible bronchoscope, it is possible to place the endotracheal tube having a desired large outer diameter without being limited by a size of an inner diameter of the laryngeal mask airway. In a case in which the tube is previously inserted into the laryngeal mask airway, even if a length of the endotracheal tube is short, the guide tube and the mask can be safely removed from the trachea without causing the endotracheal tube to be removed from the trachea.

### BRIEF DESCRIPTION OF THE DRAWINGS

FIG. 1 is an exploded perspective view illustrating a state in which a laryngeal mask airway is divided into left and right sides according to an example of the present invention.

FIG. 2 is a combined perspective view illustrating a state in which the laryngeal mask airway illustrated in FIG. 1 is integrally coupled.

FIGS. 3 to 5 are views of using states illustrating a process of intubating an endotracheal tube having a small outer diameter into the trachea using the laryngeal mask airway illustrated in FIGS. 1 and 2 of the present invention.

FIGS. 6 and 7 are exploded perspective views illustrating a state in which a laryngeal mask airway to which a modified coupling member is applied is divided into left and right sides.

FIGS. 8 to 12 are views sequentially illustrating a process of intubating an endotracheal tube having a large outer diameter into the trachea through the laryngeal mask airway and a flexible bronchoscope.

### BEST MODE FOR INVENTION

Hereinafter, preferred examples of the present invention will be described in detail with reference to the accompanying drawings. Prior to this, terms or words used in the present specification and claims should not be construed as being limited to their usual or dictionary meanings, and the inventors appropriately explain the concept of terms in order to explain their own invention in the best way. Based on the principle that it can be defined, it should be interpreted as meaning and concept consistent with the technical idea of the present invention.

Referring to the drawings, a laryngeal mask airway 100 according to an example of the present invention refers to one which is inserted into a patient's airway to secure a patient's airway passage. This laryngeal mask airway 100 serves to guide the intubation of an endotracheal tube 10 inserted into the trachea of the patient, and includes a guide tube 110, a mask 120, a handle portion 130, and a coupling member 140.

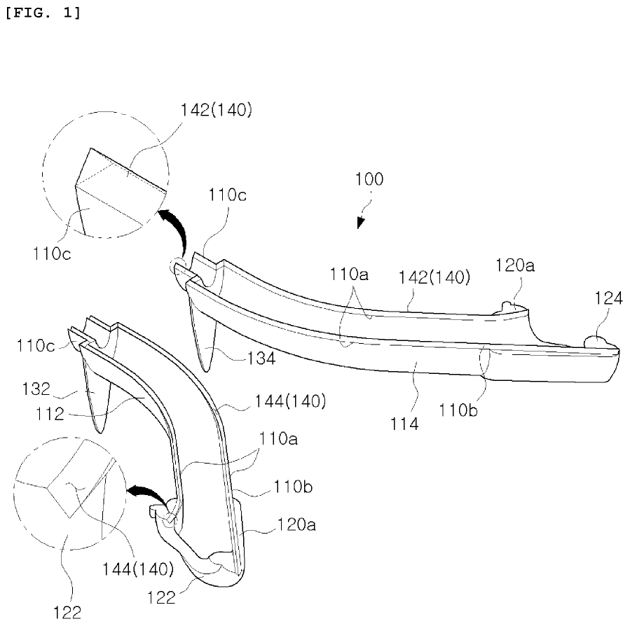

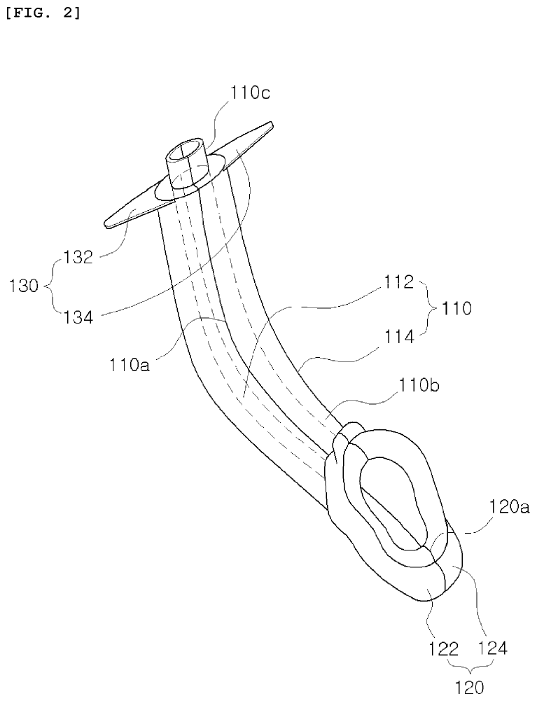

With reference to FIGS. 1 and 2, the guide tube 110 is configured such that a first cutting surface 110a is formed at a center in a longitudinal direction, an one proximal end portion 110b of the guide tube 110 is inserted into the larynx inside the trachea, and the other distal end portion 110c of the guide tube 110 is exposed to the outside of the trachea. The guide tube 110 is divided into left and right sides with respect to the first cutting surface 110a.

The guide tube 110 includes a left guide tube 112 and a right guide tube 114, and the left guide tube 112 and the right guide tube 114 are respectively disposed on the left side and the right side with respect to the first cutting surface 110a formed at the center of the guide tube 110 in the longitudinal direction.

The left guide tube 112 and the right guide tube 114 divided with respect to the first cutting surface 110a are integrally coupled by the coupling member 140 which is described later. It is preferable that the left guide tube 112 and the right guide tube 114 have corresponding shapes with respect to the first cutting surface 110a.

The mask 120 is integrally connected to the one proximal end portion 110b of the guide tube 110, serves to prevent fluid and air from flowing into the larynx inside the trachea from the outside, and is also divided into left and right sides in the same manner as the guide tube 110.

A second cutting surface 120a is formed at the center of the mask 120 in the longitudinal direction, and the mask 120 is divided to a left mask 122 and a right mask 124 with respect to the second cutting surface 120a. It is preferable that the left mask 122 and the right mask 124 are respectively disposed on the left and right sides with reference to the second cutting surface 120a, are coupled by the coupling member 140 which is described below, and are formed to correspond to each other with respect to the second cutting surface 120a.

The left guide tube 112 and the left mask 122, and the right guide tube 114 and the right mask 124 are preferably formed integrally, and the left mask 122 and the right mask 124 are preferably formed on the one proximal end portion 110b of the guide tube 110 inserted into the larynx inside the trachea.

The handle portion 130 is provided at the other distal end portion 110c of the guide tube 110 and facilitates the operator to grip the guide tube 110 so that the guide tube 110 is easily divided into left and right sides. The handle portion 130 protrudes to both sides of the guide tube 110 to correspond to each other.

It is preferable that the handle portion 130 includes a left handle 132 and a right handle 134, the left handle 132 is integrally formed with the left guide tube 112, and the right handle 134 is integrally formed with the right guide tube 114.

The guide tube 110 and the mask 120 divided into left and right sides by an external force applied by the operator are coupled by the coupling member 140, and the coupling member 140 includes a coupling protrusion 142 and a coupling groove 144. It is preferable that the coupling protrusion 142 is formed to protrude outward from the contact surface of the right guide tube 114 and the right mask 124, and the coupling groove 144 is formed to insert inward the cutting surface of the left guide tube 112 and the left mask 122.

With reference to FIG. 1, the coupling protrusion 142 preferably has a triangular shape and protrudes outward the first contact surface 110a of the right guide tube 114 and the second contact surface 120a of the right mask 124. The coupling groove 144 is preferably inserted into the first contact surface 110a of the left guide tube 112 and the second contact surface 120a of the left mask 122. The coupling protrusion 142 and the coupling groove 144 are coupled in a force-fitting method, so that when the external force is applied by the operator, the coupling protrusion 144 and the coupling groove 144 is released, and thereby the guide tube 110 and the mask 120 are divided into left and right sides.

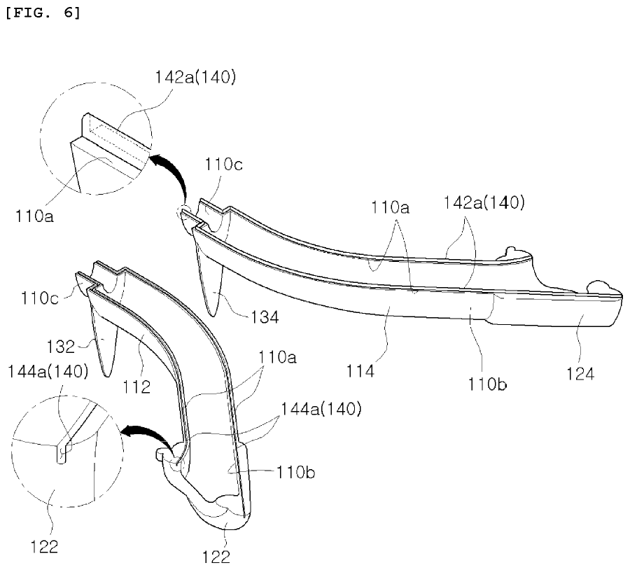

With reference to FIG. 6, the coupling protrusion 142a preferably has a shape protruding outward from the first contact surface 110a of the right guide tube 114 and the second contact surface 120a of the right mask 124. The coupling groove 144a preferably has a shape corresponding to that of the coupling protrusion 142a and is inserted into the first contact surface 110a of the left guide tube 112 and the second contact surface 120a of the left mask 122. The coupling protrusion 142a preferably has a shape other than a triangular shape, and the coupling protrusion 142a and the coupling groove 144a are coupled in the force-fitting method. Therefore, when the external force is applied by the operator, the coupling between the coupling protrusion 142a and the coupling groove 144a is released, and thereby the guide tube 110 and the mask 120 are divided into left and right sides.

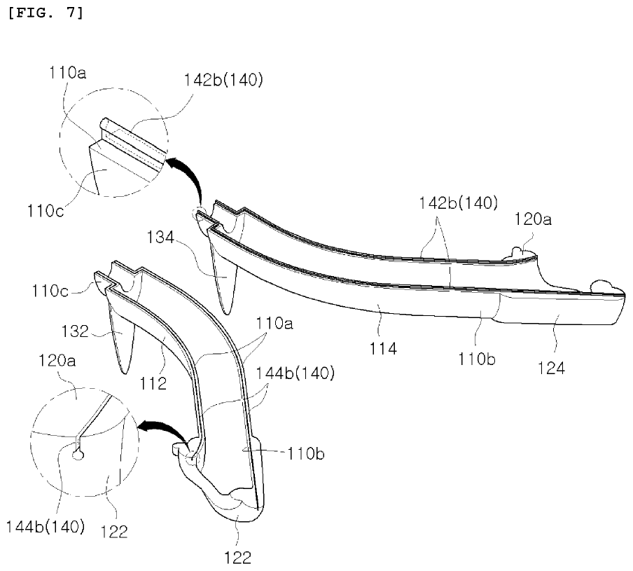

With reference to FIG. 7, the coupling protrusion 142b preferably protrudes outward from the first contact surface 110a of the right guide tube 114 and the second contact surface 120a of the right mask 124, and has a shape in which a circular protrusion is formed at an end thereof. The coupling groove 144b preferably has a shape corresponding to that of the coupling protrusion 142b, and is inserted into the first contact surface 110a of the left guide tube 112 and the second contact surface 120a of the left mask 122. The coupling protrusion 142b and the coupling groove 144b are coupled in the force-fitting method, so that when the external force is applied by the operator, the coupling of the coupling protrusion 142b and the coupling groove 144b is released and the guide tube 110 and the mask 120 are divided into the left and right sides.

The coupling protrusion 142 is formed to protrude outward from the contact surface of the right guide tube 114 and the right mask 124, and the coupling groove 144 is formed to be inserted into the left guide tube 112 and the left mask 122. However, the present invention is not limited thereto. The coupling protrusion 142 may be formed to protrude outward from the left guide tube 112 and the left mask 122, and the coupling groove 144 may be formed to be inserted into the right guide tube 114 and the right mask 124. In addition, the design may be changed so that the coupling protrusion 142 is formed on one contact surface of the left guide tube 112 and the left mask 122, and the coupling groove 144 is formed on the other contact surface thereof.

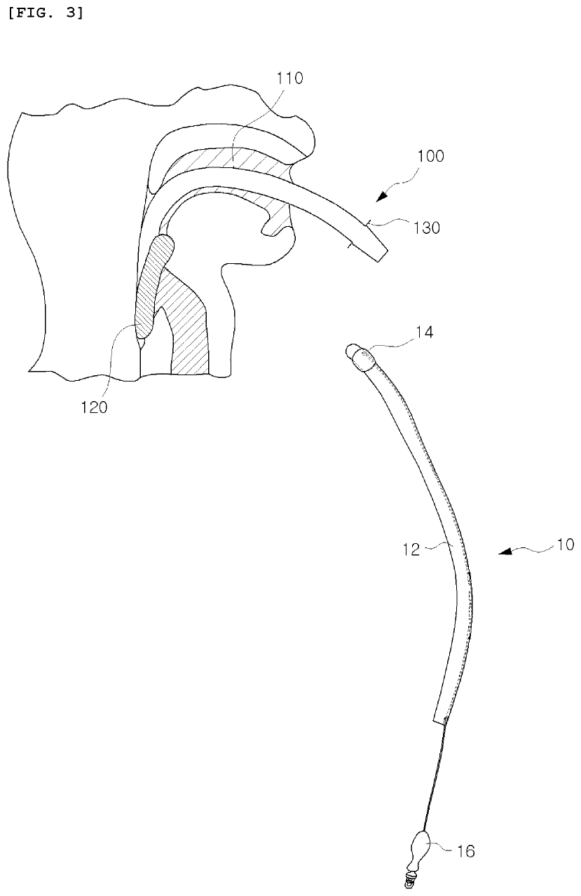

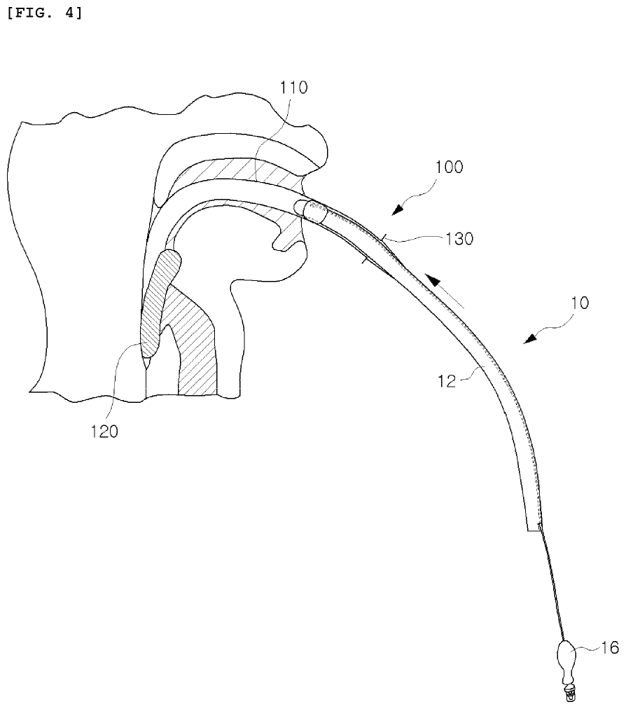

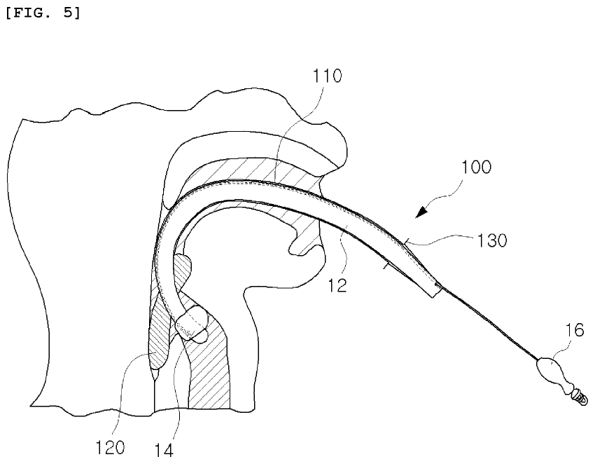

With reference to FIGS. 3 to 5, the laryngeal mask airway 100 according to the example of the present invention is intubated into the trachea to guide an endotracheal tube 10 having a small outer diameter, and the one proximal end portion 110b of the guide tube 110 including the mask 120 is preferably intubated into the trachea.

The endotracheal tube 10 includes a tube body 12, a sealing cuff 14, and a balloon member 16. It is preferable that the tube body 12 has an outer diameter smaller than the inner diameter of the guide tube 110 so that the one proximal end portion is inserted into the trachea through the guide tube 110, the other distal end portion is exposed to outside the guide tube 110, and both ends have an open shape.

The sealing cuff 14 is provided on a circumferential surface adjacent to the one proximal end portion of the tube body 12, and the sealing cuff 14 is expanded and contracted by air supplied from the outside and air discharged to the outside. The balloon member 16 is provided on the distal end portion of the tube body 12 and is connected to the sealing cuff 14 to supply and discharge air and liquid to expand and contract the sealing cuff 14. Here, the endotracheal tube 10 corresponds to a known technique, and thus a detailed description thereof will be omitted.

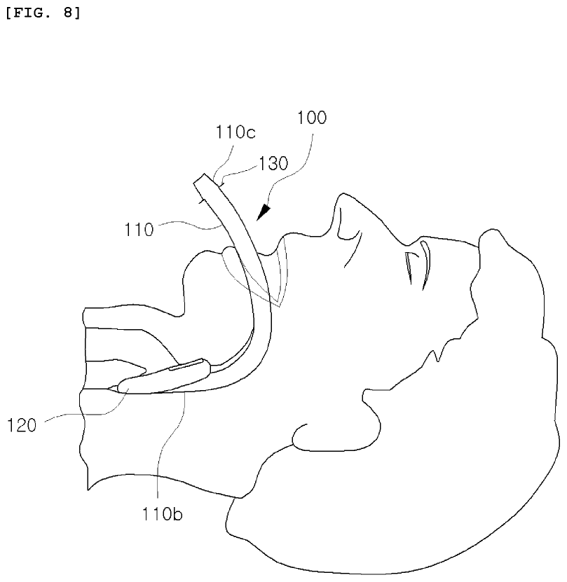

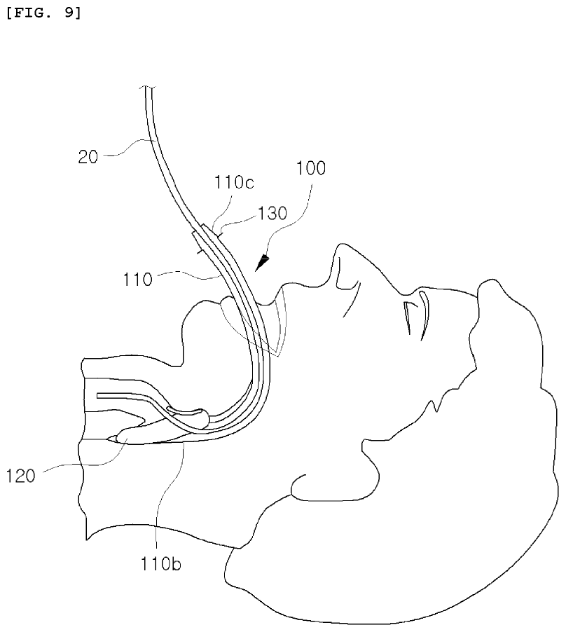

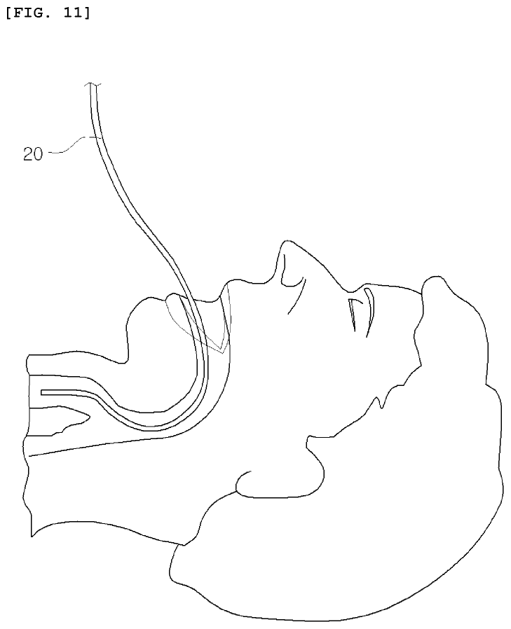

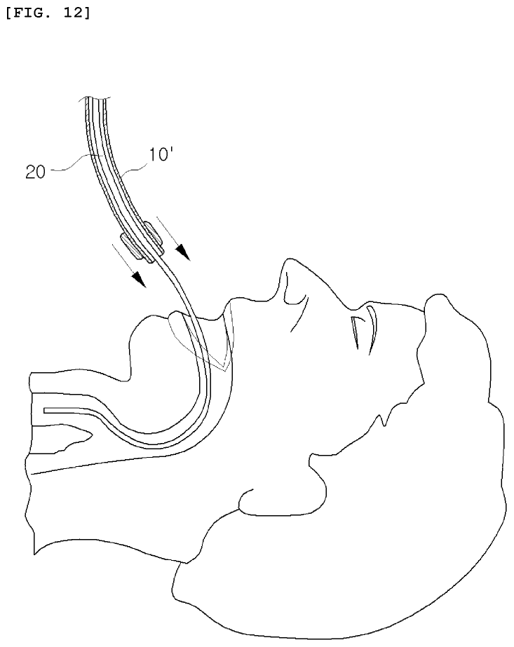

With reference to FIGS. 8 to 12, the laryngeal mask airway 100 according to the example of the present invention is also used when an endotracheal tube 10′ having an outer diameter larger than the inner diameter of the guide tube 110 is intubated, and when the endotracheal tube 10′ having a large outer diameter is intubated, the flexible bronchoscope 20 is used. With reference to FIGS. 8 and 9, the laryngeal mask airway 110 is intubated into the trachea, and the flexible bronchoscope 20 is intubated into the trachea by using the guide tube 110 of the laryngeal mask airway 100 according to the example of the present invention, and then is placed.

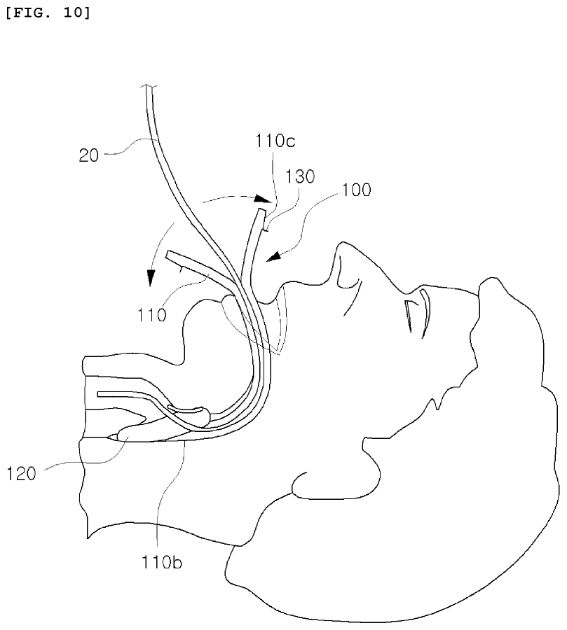

With reference to FIGS. 10 to 12, the flexible bronchoscope 20 is intubated and placed in the trachea through the guide tube 110, and then an external force is applied to the guide tube 110 and the mask 120 to divide them to the left and right sides. Thus, in a state in which the flexible bronchoscope 20 is intubated, the laryngeal mask airway 100 according to the present invention is easily separated from the trachea. After the laryngeal mask airway 100 is removed, the endotracheal tube 10′ having a large outer diameter is easily intubated into the trachea by using the flexible bronchoscope 20 that is intubated and placed in the trachea.

Therefore, after the operator grasps the handle, the guide tube 110 and the mask 120 are divided into the left and right sides with respect to the first cutting surface 110a and the second cutting surface 120a, and thereby even if the length of the endotracheal tube 10 is short, the guide tube 110 and the mask 120 can be safely removed from the trachea while the endotracheal tube 10 is not separated from the trachea.

The present invention is described with reference to the examples illustrated in the drawings, but these are only exemplary, and those of ordinary skilled in the art will appreciate that various modifications and equivalent other examples are possible therefrom. Therefore, the true technical protection scope of the present invention should be determined by the technical spirit of the appended claims.

### INDUSTRIAL APPLICABILITY

The present invention can be used in a laryngeal mask airway.

1. A laryngeal mask airway to be inserted into a patient in order to ensure that the patient's airway is open, comprising:
a guide tube that has one proximal end portion inserted into the larynx inside the trachea and the other distal end portion exposed to the outside of the trachea, guides the insertion of a flexible bronchoscope and an endotracheal tube inserted into the trachea in order to ensure that an airway is open, and is divided into left and right sides;
a mask that is integrally connected to the one proximal end portion of the guide tube, prevents fluid and air from flowing into the larynx inside the trachea from the outside, and is divided into the left and right sides;
a handle portion that is provided on the other side of the guide tube and protrudes to both sides of the guide tube to correspond to each other such that an operator easily holds the guide tube; and
a coupling member that couples the guide tube and mask, which are divided into the left and right sides, to each other.

2. The laryngeal mask airway according to claim 1,
wherein a first cutting surface is formed at a center of the guide tube in a longitudinal direction,
wherein the guide tube includes a left guide tube disposed on a left side with respect to the first cutting surface, and a right guide tube disposed on a right side with respect to the first cutting surface,
wherein the left guide tube and the right guide tube are coupled and detached by the coupling member, and
wherein the left guide tube and the right guide tube have corresponding shapes.

3. The laryngeal mask airway according to claim 1,
wherein a second cutting surface is formed at a center of the mask in a longitudinal direction,
wherein the mask includes a left mask disposed on a left side with respect to the second cutting surface, and a right mask disposed on a right side with respect to the second cutting surface,
wherein the left mask and the right mask are coupled and detached by the coupling member, and
wherein the left mask and the right mask have corresponding shapes.

4. The laryngeal mask airway according to claim 2,
wherein the left guide tube and the left mask are integrally formed, and the right guide tube and the right mask are integrally formed, and
wherein the coupling member includes a coupling protrusion formed on a contact surface of the right guide tube and the right mask, and a coupling groove formed on a contact surface of the left guide tube and the left mask, and on which the coupling protrusion is mounted.

5. The laryngeal mask airway according to claim 4,
wherein the coupling protrusion has a triangular shape and protrudes outward from the contact surface of the right guide tube and the right mask, and
wherein the coupling groove has a shape corresponding to that of the coupling protrusion, and is inserted into the contact surface of the left guide tube and the left mask.

6. The laryngeal mask airway according to claim 4,
wherein the coupling protrusion has a shape protruding outward from the contact surface of the right guide tube and the right mask, and
wherein the coupling groove has a shape corresponding to that of the coupling protrusion, and is inserted into the contact surface of the left guide tube and the left mask.

7. The laryngeal mask airway according to claim 4,
wherein the coupling protrusion protrudes outward from the contact surface of the right guide tube and the right mask, and has a shape in which a circular protrusion is formed at an end, and
wherein the coupling groove has a shape corresponding to that of the coupling protrusion, and is inserted into the contact surface of the left guide tube and the left mask.

8. The laryngeal mask airway according to claim 3,
wherein the left guide tube and the left mask are integrally formed, and the right guide tube and the right mask are integrally formed, and
wherein the coupling member includes a coupling protrusion formed on a contact surface of the right guide tube and the right mask, and a coupling groove formed on a contact surface of the left guide tube and the left mask, and on which the coupling protrusion is mounted.
